(19) United States
(12) Patent Application Publication
(10) Pub. No.: US 2015/0011415 A1
(43) Pub. Date: Jan. 8, 2015

(54) METHOD FOR CALCULATING A DISEASE RISK SCORE

(71) Applicant: Imperial Innovations Limited, London (GB)

(72) Inventors: Michael Levin, London (GB); Myrsini Kaforou, London (GB); Jethro Herberg, London (GB); Victoria Wright, London (GB); Lachlan Coin, London (GB)

(21) Appl. No.: 14/376,014

(22) PCT Filed: Feb. 1, 2013
(86) PCT No.: PCT/GB2013/050225

§ 371 Date: Jul. 31, 2014

(30) Foreign Application Priority Data
Feb. 1, 2012 (GB) 1201766.1

(51) Int. Cl. G06F 19/20; C12Q 1/70

(52) U.S. Cl. CPC G06F 19/20; C12Q 1/701
USPC 506/9; 506/17; 702/19

(57) ABSTRACT

The present disclosure relates to a general method for converting complex gene expression data into a simple, composite disease risk score which can be used for the development of rapid diagnostic tests suitable for clinical use for the determination of the presence of an infection or disease in a host.

The present disclosure relates to a general method for converting complex gene expression data into a simple, composite disease risk score which can be used for the development of rapid diagnostic tests suitable for clinical use and to kits comprising one or more elements employed in the method.

### BACKGROUND

The simultaneous measurement of whole genome RNA expression by microarray and RNA-seq techniques has provided powerful methods for analysing expression of genes. There is clear evidence that many diseases and biological processes are characterised by distinct patterns of RNA expression which can be detected by microarray analysis. RNA expression “signatures” have been described for many diseases and disease stages in which complex patterns of RNA expression of multiple gene transcripts allow distinction between the patients affected by a disease and healthy controls or patients with other diseases. Disease signatures have been reported for several infectious diseases including malaria (1), meningococcal infection (2), immunodeficiencies (3), viral infections (4), TB (5), cancer (6) and inflammatory diseases (7).

Although the published literature on the use of RNA expression microarrays suggests that diagnosis using gene expression signatures has great clinical potential, its application in disease diagnosis has been limited by the complexity of the microarray analysis process, the requirement for sophisticated array scanning technology, the need for advanced bioinformatic analysis and the overall cost of the methodology. In order for the clear biological information provided by microarray signatures to be routinely utilised for clinical diagnosis, new methods are required which will enable complex microarray signatures of disease to be converted into simple diagnostic tests which do not rely on sophisticated equipment or complex bioinformatic analysis, and which can be developed as simple, affordable, near patient assays suitable for clinical use, even in low resource settings.

Described herein is a novel method to convert complex multi-transcript gene expression signatures into a simple composite disease risk score. Furthermore we describe how this method can be used to provide simplified diagnostic tests for disease signatures which are suitable for wide clinical use even in low resource settings. We also demonstrate use of the method in generating a signature and score for Influenza H1N1.

### SUMMARY OF THE METHOD

The present disclosure provides a method of processing gene expression data generated from analysis of an ex vivo patient-sample, for example for establishing the presence of a signature, for example a predefined signature, indicative of infection by a pathogen, or specific to an inflammatory, malignant or other defined disease comprising the steps:
a) optionally normalising and/or scaling numeric values of the gene expression data
b) taking the normalised and/or scaled numeric values or the raw numeric values, each of which comprise both positive and/or negative numeric values and designating all said numeric values to be negative or alternatively all positive,
c) optionally refining the discriminatory power of one or more up-regulated genes and down-regulated genes by statistically weighting some of the numeric values associated therewith, and
d) summating the positive or negative numeric values obtained from step b) or step c) to provide a composite expression score,
   wherein the composite expression score obtained from step d) is compared to a control and the comparison allows the sample to be designated as positive or negative for the relevant infection or disease.

The method is broadly applicable to any disease or biological process for which a multi-gene signature can be or has been identified for example using RNA or DNA expression including and inflammatory, chronic diseases or malignant conditions which are defined by specific clinical diagnostic criteria. In one embodiment the method is suitable for establishing a signature indicative of infection by a pathogen. Advantageously it provides a single value that can readily be characterised as positive for the disease or infection. Advantageously this allows patients with an infection to be discriminated from those without the infection. Advantageously it provides a single value that can be used to distinguish patients with an active disease or infection from those with latent or inactive disease or infection.

The methods of the present disclosure are advantageous in that they allow the deployment of gene expression profiles for routine clinical testing, in a rapid, cost efficient and robust way, for example to diagnose bacterial infection or viral infection. This allows patients to be rapidly given appropriate treatment, such as antibiotics in the case of bacterial infection and in the case of acute viral infection, once shown to be negative for bacterial infection, an antipyretic can be given and further investigation may be avoided. Given the fact that the emergence of antibiotic resistance of bacteria to antibiotics is becoming a significant problem the present methods allows inappropriate administration of antibiotic treatment to be minimised.

In addition the methods of the present disclosure are sufficiently sensitive to distinguish subtle differences in the diseases and/or infections in patients, even in the presence of complicating factors, such as underlying disease, such as HIV or malaria.

In places such as sub-Saharan Africa this rapid and effective diagnosis is likely to save lives and ensure that precious resources are used where they are needed most.

### BRIEF DESCRIPTION OF THE FIGURES

FIG. 1: Heat map showing unsupervised clustering of Influenza H1N1 cases from controls. Each column corresponds to a sample and each line corresponds to a transcript. Darker shades reflect over-expression while lighter shades reflect under-expression. H1N1 samples are shown with an arrow and controls are also labelled with an arrow.

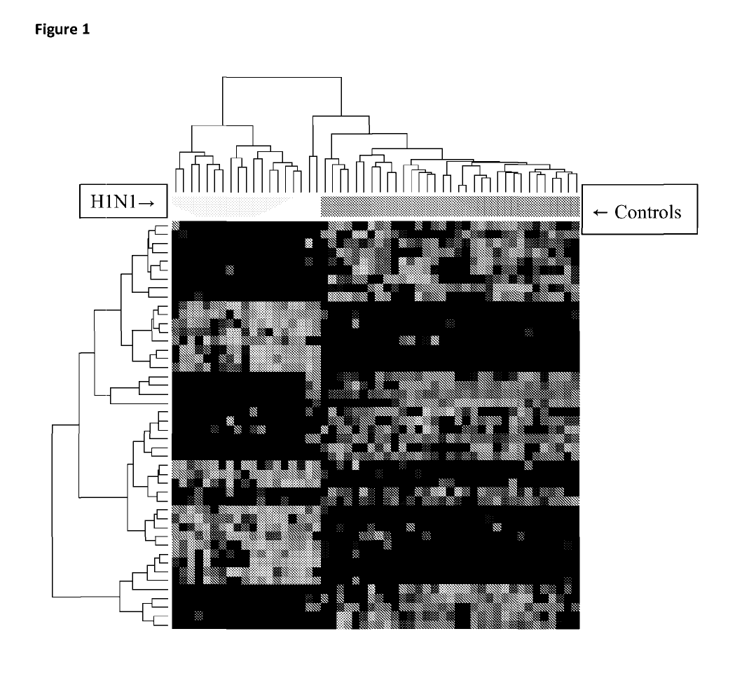

FIG. 2: Total fluorescence of H1N1 vs controls. Means and 25^{th} and 75^{th} percentile are shown. Boxes shows the sensitivity and specificity, positive and negative predictive value of the total fluorescence score

FIG. 3: Weighting of the transcripts improves discrimination of H1N1 vs control

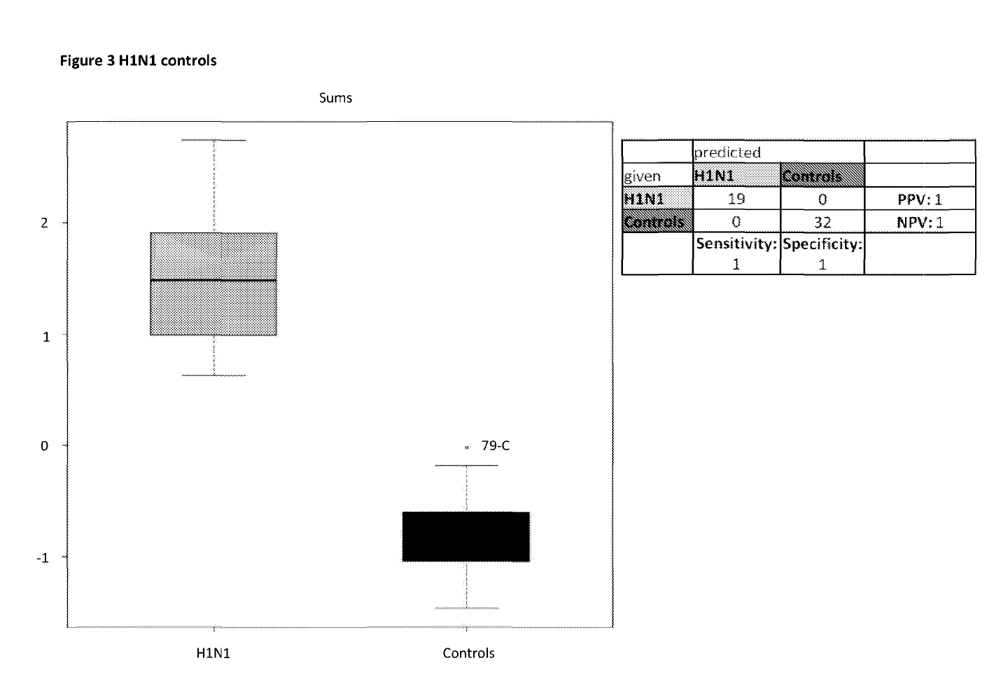

FIG. 4: Discrimination of H1N1 from RSV using the total fluorescence score

FIG. 5: Improved discrimination of H1N1 from RSV infected patients using the weighting of transcripts

FIG. 6: Application of the total fluorescence score to patients with H1N1, RSV, bacterial infection, other viruses, and unclassified ill patients without detected pathogens

FIG. 7: Shows the top canonical pathways differing between H1N1/09 and controls, RSV and Bacterial infection. Each bar is filled in proportion to the number of DE H1N1/09 transcripts increased (diagonal stripes) or decreased (grey) in abundance relative to the comparator cohort. The total bar length is proportional to P value. Patterned blocks next to each pathway are coded according to biological function. Protein synthesis pathways (horizontal stripes) were the most significant in all 3 comparisons, with predominant decreased expression in H1N1/09 patients relative to the comparator group. Innate immune pathway transcripts (vertical stripes) were increased in H1N1/09 patients, whilst adaptive immune transcripts (black) were reduced relative to controls.

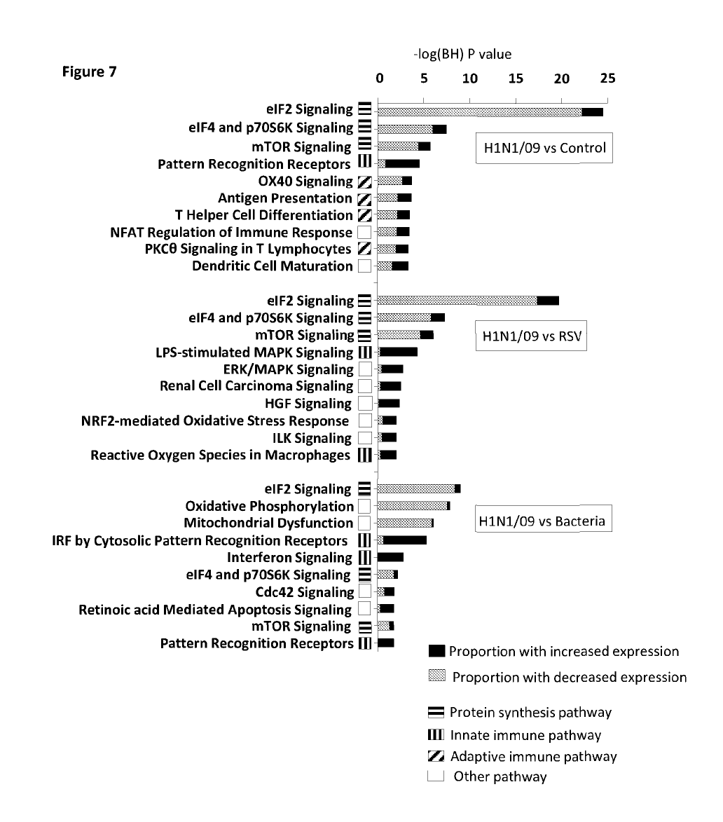

### DETAILED DESCRIPTION OF THE DISCLOSURE

In one embodiment the method is used to generate a composite expression score. The composite expression score can be used to designate a sample as positive or negative for infection or disease.

In one embodiment the method is used to generate an individual's composite expression score which can then be used to diagnose infection or disease.

Gene expression data as employed herein is intended to refer to any data generated from a patient sample that is indicative of the expression of the two or more genes, for example 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61m 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300 or the whole genome.

It is important to appreciate that the gene expression measured is that of the host (e.g. human) not that of the infectious agent or disease.

In one embodiment the gene profile is the minimum required to detect the infection or discriminate the disease. In one embodiment the minimal disease specific transcript is specific to a virus. In one embodiment the minimal disease specific transcript is specific to bacteria. In one embodiment the minimal disease specific transcript is specific to Gram positive bacteria. In one embodiment the minimal disease specific transcript is specific to Gram negative bacteria. In one embodiment the minimal disease specific transcript is specific to a fungus. In one embodiment the minimal disease specific transcript is specific to a parasite.

Specific to a virus as employed herein means specific to a host infected with a virus.

Specific to bacteria as employed herein means specific to a host infected with bacteria.

Specific to Gram positive bacteria as employed herein means specific to a host infected with Gram positive bacteria.

Specific to Gram negative bacteria as employed herein means specific to a host infected with Gram negative bacteria.

Specific to a fungus as employed herein means specific to a host infected with a fungus.

Specific to a parasite as employed herein means specific to a host infected with a parasite.

In one embodiment the gene profile is specific for an inflammatory disease such as rheumatoid arthritis, Kawasaki disease, Still's diseases or multiple sclerosis

In one embodiment the gene profile is specific for malignant diseases, for example cancer such as lung cancer, breast cancer, colon cancer, bowel cancer, prostate cancer, liver cancer, melanoma or similar or a chronic non-infectious diseases, such as autoimmune disease (e.g. ulcerative colitis, lupus erythematosus, Crohn's disease and Coeliac disease) and graft versus host disease.

In one embodiment the gene expression data is generated from a micro array, such as a gene chip.

Microarray as employed herein includes RNA or DNA arrays, such as RNA arrays.

A gene chip is essentially a microarray that is to say an array of discrete regions, typically nucleic acids, which are separate from one another and are typically arrayed at a density of between, about 100/cm² to 1000/cm², but can be arrayed at greater densities such as 10000/cm².

The principle of a microarray experiment, is that mRNA from a given cell line or tissue is used to generate a labelled sample typically labelled cDNA or cRNA, termed the ‘target’, which is hybridized in parallel to a large number of, nucleic acid sequences, typically DNA or RNA sequences, immobilised on a solid surface in an ordered array. Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 microns in size and are spaced about the same distance apart.

Using this technique, arrays consisting of more than 30,000 cDNAs can be fitted onto the surface of a conventional microscope slide. For oligonucleotide arrays, short 20-25mers are synthesized in situ, either by photolithography onto silicon wafers (high-density-oligonucleotide arrays from Affymetrix or by ink-jet technology (developed by Rosetta Inpharmatics, and licensed to Agilent Technologies). Alternatively, pre-synthesised oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of pre-synthesised longer oligonucleotides (50-100mers) has recently been developed to counteract these disadvantages.

In one embodiment the gene expression data is generated in solution using appropriate probes for the relevant genes.

In one embodiment the gene chip is an off the shelf gene chip commercially available chip, for example HumanHT-12 v4 Expression BeadChip Kit, available from Illumina, NimbleGen microarrays from Roche, Agilent, Eppendorf and Genechips from Affymetrix such as HU-UI 33.Plus 2.0 gene chips.

In an alternate embodiment the gene chip is a bespoke gene chip, that is to say the chip contains only the target genes which are relevant to the desired profile. Custom made chips can be purchased from companies such as Roche, Affymetrix and the like. In yet a further embodiment the bespoke gene chip comprises a minimal disease specific transcript set.

In one embodiment the method according to the present disclosure and for example chips employed therein may comprise one or more house-keeping genes. House-keeping genes as employed herein is intended to refer to genes that are not directly relevant to the profile for identifying the disease or infection but are useful for statistical purposes and/or quality control purposes, for example they may assist with normalising the data, in particular a house-keeping gene is a constitutive gene i.e. one that is transcribed at a relatively constant level. The housekeeping gene's products are typically needed for maintenance of the cell. Examples include actin, GAPDH and ubiquitin.

In one or more embodiments, the method and chips employed therein may include use of one or more genes native to a pathogen or relevant to the disease, for example to assist or confirm the results of the analysis.

The present disclosure extends to a custom made chip comprising a minimal discriminatory gene set for diagnosis of infection by a pathogen, or diagnosis of inflammatory or other specific diseases, for example employing a gene profile identified by a method described below.

Thus in one embodiment DNA or RNA from the patient sample, (which may be blood, tissue or other cell containing fluid) is analysed.

In one or more embodiments the analysis is ex vivo.

In one embodiment the gene chip is a fluorescent gene chip that is to say the readout is fluorescence.

Fluorescence as used herein means the emission of light by a substance that has absorbed light or other electromagnetic radiation.

In an alternate embodiment the gene chip is a colorimetric gene chip, for example colorimetric gene chip uses microarray technology wherein avidin is used to attach enzymes such as peroxidase or other chromogenic substrates to the biotin probe currently used to attach fluorescent markers to DNA. The present disclosure extends to a microarray chip adapted to read by colorimetric analysis and adapted for the analysis of infection in a patient sample. The present disclosure also extends to use of a colorimetric chip to analyse a patient sample for infection, in particular an infection defined herein.

Colorimetric means a test based on colour perception.

In an alternative embodiment, a gene set indicative of the disease under investigation may be detected by physical detection methods including nanowire technology, changes in electrical impedance, or microfluidics.

Thus for application of disease signatures in low resource settings or for rapid diagnosis in near patient tests the readout for the assay can be converted from a fluorescent readout as used in current microarray technology into a simple colorimetric format or one using physical detection methods such as changes in impedance, which can be read with minimal equipment. For example, this is achieved by utilising the Biotin currently used to attach fluorescent markers to DNA. Biotin has high affinity for avidin which can be used to attach enzymes such as peroxidase or other chromogenic substrates. This process will allow the quantity of cRNA binding to the target transcripts to be quantified using a chromogenic process rather than fluorescence. Simplified assays providing yes/no indications of disease status can then be developed by comparison of the colour intensity of the up- and down-regulated pools of transcripts with control colour standards. Similar approaches can enable detection of multiple gene signatures using physical methods such as changes in electrical impedance.

The methods employing colorimetric readouts are likely to be particularly advantageous for use in remote or under resourced places, for example Africa because the equipment required to read the chip is likely to be simpler.

In one embodiment the method of the present disclosure is employed for detection of infection by a pathogen, for example a virus or bacteria.

Pathogen as used herein is microorganism that causes disease in its host.

In one embodiment there is provided a method to determine whether an infection is viral, bacterial, parasitic or fungal.

In one embodiment the method according to the present invention may be employed to detect a viral infection for example, Influenza such as Influenza A, including but not limited to: H1N1, H2N2, H3N2, H5N1, H7N7, H1N2, H9N2, H7N2, H7N3, H10N7, Influenza B and Influenza C, Respiratory Syncytial Virus (RSV), rhinovirus, enterovirus, bocavirus, parainfluenza, adenovirus, metapneumovirus, herpes simplex virus, Chickenpox virus, Human papillomavirus, Hepatitis, Epstein-Barr virus, Varicella-zoster virus, Human cytomegalovirus, Human herpesvirus, type 8 BK virus, JC virus, Smallpox, Parvovirus B19, Human astrovirus, Norwalk virus, coxsackievirus, poliovirus, Severe acute respiratory syndrome virus, yellow fever virus, dengue virus, West Nile virus, Rubella virus, Human immunodeficiency virus, Guanarito virus, Junin virus, Lassa virus, Machupo virus, Sabia virus, Crimean-Congo haemorrhagic fever virus, Ebola virus, Marburg virus, Measles virus, Mumps virus, Rabies virus, Rotavirus

In one embodiment the method according to the present disclosure may be employed to detect a bacterial infection, such as Chlamydia pneumoniae, Chlamydia trachomatis, Chlamydophila psittaci, Mycoplasma pneumonia.

In one embodiment the method according to the present disclosure may be employed to detect a Gram positive bacterial infection, such as but not limited to Corynebacterium diphtheriae, Clostridium botulinum, Clostridium difficile, Clostridium perfringens, Clostridium tetani, Enterococcus faecalis, Enterococcus faecium, Listeria monocytogenes, Staphylococcus aureus, Staphylococcus epidermidis, Staphylococcus saprophyticus, Streptococcus agalactiae, Streptococcus pneumoniae, Streptococcus pyogenes, or acid fast bacteria such as Mycobacterium leprae, Mycobaterium tuberculosis, Mycobacterium ulcerans and mycobacterium avium intercellularae

In one embodiment the method according to the present disclosure may be employed to detect a Gram negative bacterial infection, such as but not limited to Bordetella pertussis, Borrelia burgdorferi, Brucella abortus, Brucella canis, Brucella melitensis, Brucella suis, Campylobacter jejuni, Escherichia coli, Francisella tularensis, Haemophilus influenzae, Helicobacter pylori, Legionella pneumophila, Leptospira interrogans, Neisseria gonorrhoeae, Neisseria meningitidis, Pseudomonas aeruginosa, Rickettsia rickettsii, Salmonella typhi, Salmonella typhimurium, Shigella sonnei, Treponema pallidum, Vibrio cholerae, Yersinia pestis.

In one embodiment the method according to the present disclosure may be employed to detect a parasite such as protozoa, helminths and ectoparasites, including, but not limited to Entamoeba histolytica, Plasmodium Sp. Trypanosoma brucei, Giardia lamblia, Ancylostoma, Ascaris, Brugia, Wuchereria, Onchocerca, Schistosoma, Trichuris and malaria.

In one embodiment the method according to the present disclosure may be employed to detect a fungus such as Candida, Aspergillus, Cryptococcus, Histoplasma, Pneumocystis and Stachybotrys species.

In one embodiment the method is employed to detect tuberculosis including latent tuberculosis, and distinguish tuberculosis from other conditions with similar clinical features.

In one embodiment the method according to the present disclosure is performed on a patient with acute infection.

In a further embodiment the patient-sample is from a febrile patient, that is to say with a temperature above the normal body temperature of 37.5° C.

In yet a further embodiment the analysis is performed to establish if a fever is associated with a bacterial or viral infection. Establishing the source of the fever/infection advantageously allows the prescription and/or administration of appropriate medication, for example those with bacterial infections can be given antibiotics and those with viral infections can be given antipyretics.

Efficient treatment is advantageous because it minimises hospital stays, ensures that patients obtain appropriate treatment, which may save lives, especially when the patient is an infant or child, and also ensures that resources are used appropriately.

In recent years it has become apparent that the over-use of antibiotics should be avoided because it leads to bacteria developing resistance. Therefore, the administration of antibiotics to patients who do not have bacterial infection should be avoided.

In addition the method may be employed to identify which subcategory the infection falls into and therefore provide information which assists in selecting the specific treatment.

In other embodiments the method may be used to facilitate diagnosis of a range of inflammatory and neoplastic diseases including but not limited to SLE, Kawasaki disease, rheumatoid arthritis, Still's disease, Crohn's disease, sarcoidosis, multiple sclerosis, polyarteritis, disseminated carcinoma, lymphoma.

Such conditions are diagnosed using specific clinical diagnostic criteria. These are criteria commonly known and used by doctors to determine infection or disease and to specify one infection or disease from another infection or disease.

Normalising as employed herein is intended to refer to statistically accounting for background noise by comparison of data to control data, such as the level of fluorescence of house-keeping genes, for example fluorescent scanned data may be normalized using RMA to allow comparisons between individual chips. The following reference describes this method. Irizarry et al (21).

Scaling as employed herein refers to boosting the contribution of genes which are expressed at low levels or have a high fold change but still relatively low fluorescence such that their contribution to the diagnostic signature is increased.

Fold change is often used in analysis of gene expression data in microarray and RNA-Seq experiments, for measuring change in the expression level of a gene and is calculated simply as the ratio of the final value to the initial value i.e. if the initial value is A and final value is B, the fold change is B/A Tusher et al (22).

In programs such as Arrayminer, fold change of gene expression can be calculated. The statistical value attached to the fold change is calculated and is the more significant in genes where the level of expression is less variable between patients in different groups and, for example where the difference between groups is larger

Patient-sample as employed herein is a sample from any person with or without a disease including a person suspected disease from whom a sample has been collected. A patient derived sample includes a positive or negative control employed in the method.

The step of obtaining a suitable sample from the patient is a routine technique, which involves taking a blood sample. This process presents little risk and does not need to be performed by a doctor but can be performed by appropriately trained support staff. In one embodiment the sample derived from the patient is approximately 2 ml of blood, however smaller volumes can be used for example 0.5-1 mI. Blood or other tissue fluids are immediately placed in an RNA stabilizing buffer such as included in the Pax gene tubes, or Tempus tubes.

If storage is required then it should usually be frozen within 3 hours of collections at approximately −70° C.

In one embodiment the gene expression data is generated from RNA levels in the sample.

For microarray analysis the blood may be processed using a suitable product, such as PAXgene blood RNA extraction kits (Qiagen).

Total RNA may also be purified using the Tripure method—Tripure extraction (Roche Cat. No. 1 667 165). The manufacturers protocols may be followed. This purification may then be followed by the use of an RNeasy Mini kit—clean-up protocol with DNAse treatment (Qiagen Cat. No. 74106).

Quantification of RNA may be completed using optical density at 260nm and Quant-IT RiboGreen RNA assay kit (Invitrogen—Molecular probes RI 1490). The Quality of the 28s and 18s ribosomal RNA peaks can be assessed by use of the Agilent bioanalyser.

In another embodiment the method further comprises the step of amplifying the RNA. Amplification may be performed using a suitable kit, for example TotalPrep RNA Amplification kits (Applied Biosystems).

In one embodiment an amplification method may be used in conjunction with the labelling of the RNA for microarray analysis. The Nugen 3′ ovation biotin kit (Cat: 2300-12, 2300-60).

The RNA derived from the patient sample is then hybridised to the relevant probes, for example which may be located on a chip. After hybridisation and washing, where appropriate, analysis with an appropriate instrument is performed.

In performing an analysis to ascertain whether a patient presents with a gene signature indicative of disease or infection according to the present disclosure, the following steps are performed: obtain mRNA from the sample and prepare nucleic acids targets, hybridise to the array under appropriate conditions, typically as suggested by the manufactures of the microarray (suitably stringent hybridisation conditions such as 3×SSC, 0.1% SDS, at 50<0>C) to bind corresponding probes on the array, wash if necessary to remove unbound nucleic acid targets and analyse the results.

In one embodiment the readout from the analysis is fluorescence.

In one embodiment the readout from the analysis is colorimetric.

In one embodiment all of the up-regulated genes are physically located in close proximity on the diagnostic test, for example in a well or on a chip or equivalent.

In one embodiment all of the down-regulated genes are physically located in close proximity on the diagnostic test, for example in a well or on a chip or equivalent.

In one embodiment all of the up-regulated genes are physically distant or separated from all of the down-regulated genes on the diagnostic test, for example in separate wells or spots.

In one embodiment physical detection methods such as changes in electrical impedance, nanowire technology or microfluidics may be used.

In one embodiment there is provided a method which further comprises the step of quantifying RNA from the patient-sample.

If a quality control step is desired, software such as Genome Studio software may be employed.

Numeric value as employed herein is intended to refer to a number obtained for each relevant gene from the analysis or readout of the gene expression, for example the fluorescence or colorimetric analysis. The numeric value obtained from the initial analysis may be manipulated, corrected and if the result of the processing is a still a number then it will be continue to be a numeric value.

By “converting” is meant processing of a negative numeric value to make it into a positive value or processing of a positive numeric value to make it into a negative value by simple conversion of a positive sign to a negative or vice versa.

Up-regulated as employed herein is intended to refer to a gene transcript which is expressed at higher levels in a diseased or infected patient-sample relative to a control-sample free from a relevant disease or infection, or in a latent or different stage of the infection

Down-regulated as employed herein is intended to refer to a gene transcript which is expressed at lower levels in a diseased or infected patient-sample relative to a control-sample free from a relevant disease or infection.

Analysis of the patient-derived sample will, for the genes analysed, give a range of numeric values some of which are positive (preceded by+ and in mathematical terms considered greater than zero) and some of which are negative (preceded by and in strict mathematical terms are considered to less than zero). The positive and negative in the context of gene expression analysis is a convenient mechanism for representing genes which are up-regulated and genes which are down regulated.

In the method of the present disclosure either all the numeric values of genes which are down-regulated and represented by a negative number are converted to the corresponding positive number (i.e. by simply changing the sign) for example −1 would be converted to 1 or all the positive numeric values for the up-regulated genes are converted to the corresponding negative number.

The present inventors have established that this step of rendering the numeric values for the gene expressions positive or alternatively all negative allows the summating of the values to obtain a single value that is indicative of the presence of disease or infection or the absence of the same.

This is a huge simplification of the processing of gene expression data and represents a practical step forward thereby rendering the method suitable for routine use in the clinic.

Surprisingly this single value is able to discriminate for the presence of an infection or disease.

By discriminatory power is meant the ability to distinguish between an infected and a non-infected sample or between a given infection and other infections or between a latent infection and an active infection or between patients with a specified inflammatory or non-infectious disease and other conditions with similar symptoms.

The discriminatory power of the method according to the present disclosure may, for example be increased by attaching more weighting to genes which are more significant in the profile, even if they are expressed at low or lower absolute levels.

As employed herein, raw numeric value is intended to, for example refer to unprocessed fluorescent values from the gene chip, either absolute fluorescence or relative to a house keeping gene or genes.

Summating as employed herein is intended to refer to the act or process of adding numerical values.

Composite expression score as employed herein means the sum (aggregate number) of all the individual numerical values generated for the relevant genes by the analysis, for example the sum of the fluorescence data for all the relevant up and down regulated genes. The score may or may not be normalised and/or scaled and/or weighted.

Composite expression score, simple score, simple composite disease risk score, single value, single disease risk score are used interchangeably throughout the description and refer to the number output from the method described herein. Where the total fluorescence (up or down-regulated) is summated for the gene profile.

In one embodiment the composite expression score is normalised.

In one embodiment the composite expression score is scaled.

In one embodiment the composite expression score is weighted.

Weighted as employed herein is intended to refer to the relevant value being adjusted to more appropriately reflect its contribution to the profile.

Control as employed herein is intended to refer to a positive (control) sample and/or a negative (control) sample which, for example is used to compare the patient sample to, and/or a numerical value or numerical range which has been defined to allow the patient sample to be designated as positive or negative for disease/infection by reference thereto.

Positive control sample as employed herein is a sample known to be positive for the pathogen or disease in relation to which the analysis is being performed.

Negative control sample as employed herein is intended to refer to a sample known to be negative for the pathogen or disease in relation to which the analysis is being performed.

In one embodiment the control is a sample, for example a positive control sample or a negative control sample, such as a negative control sample.

In one embodiment the control is a numerical value, such as a numerical range, for example a statistically determined range obtained from an adequate sample size defining the cut-offs for accurate distinction of disease cases from controls.

In one embodiment the signature indicative of disease or infection is a predefined signature.

Signature indicative of disease or infection means the minimum genes required to determine the presence of a given infection.

Predefined signature as employed herein is intended to refer to a signature that comprises a defined set of genes where in a specific number thereof are up-regulate and/or down-regulated in the presence of disease or infection.

Predetermined profile means the profile of genes that are up and/or down-regulated in the infected or diseased host.

Predefined signature, predetermined profile, Gene profile, specific gene expression profile, minimal disease specific transcript set, minimal discriminatory gene set, minimal disease-specific gene set, minimum transcript number, minimal transcript set and minimal discriminatory gene list are used to refer to the same set of genes or transcripts. That is, the minimum set required to determine a given infection. Typically these terms encompass the maximally discriminatory transcripts.

The generation of the relevant gene lists can be performed using an appropriate statistical analysis tool, for example elastic net which simultaneously handles automatic variable selection and continuous shrinkage, and it can select groups of correlated variables. The method is explained in Zou et al (8). The relevant algorithms of the fully functioning elastic net are incorporates herein by reference.

“Using the Elastic Net Coefficients” Approach

Variable selection methods, such as elastic net, provide coefficients that represent the contribution of every transcript towards a good classification of the samples. The “coefficients weighted” expression values are a result of multiplying the expression values not by +1 and −1. according to the fold change of the transcripts in the groups, but by their coefficients. Coefficients' signs are calculated according to the positive or negative fold change.

Alternative methods for generating gene lists include Lasso, Hyperlasso, Spotfire Analysis, Baldi BH analysis and Arrayminer analysis or a combination of at least two (such as three or four) of the methods described herein.

The following step may be followed to identify a gene list or profile suitable for discriminating if a patient has an infection with a pathogen.

Step 1: Identification of Differentially Expressed (DE) Transcripts and Genes that Distinguish Disease or Condition of Interest from Comparator Diseases or Healthy Controls.

The first step in the development of a disease specific marker according to the present disclosure is to undertake a microarray analysis in which a cohort of patients with the specific disease under study are compared with comparator groups unaffected by the disease, and/or affected by other diseases which require discrimination from the disease under study, and/or with a latent infection with the specific disease under study. Numerous publications adequately describe the process of identifying gene signatures of disease processes, including the need for adequate sample size, data quality control and the use of independent cohorts: one for initial discovery of the gene signature and another for validation of the identified signature (4,6,7). After identifying differentially expressed RNA transcripts that distinguish between cases and controls, further analysis is required to identify the minimal disease-specific gene set.

Step 2: Identification of the Minimal Disease Specific Set of Transcripts

For many disease processes, a very large number of differentially expressed RNA transcripts between cases and comparator groups can be identified by modified parametric statistical tests, after multiple hypothesis correction. In order to identify the minimum transcript number required for disease classification variable selection using published algorithms performed, for example employing elastic net for RNA analysis in combination with cross-validation to reduce over-fitting (8). Other adequate variable selection methods can be also used (e.g. Lasso, Hyperlasso). In this way, a disease signature containing thousands of RNA transcripts can be reduced to a much smaller number (for instance <50) of maximally discriminatory transcripts. The performance of the minimal transcript set at distinguishing disease cases from others is assessed by validation on independent cohorts.

In one embodiment there is provided a method of identifying a gene list or profile suitable for discriminating if a patient has an infection with a pathogen or a disease comprising step 1 and step 2.

In one embodiment the present disclosure extends to a gene list indicative of infection by a pathogen, such as a virus or bacteria, in particular bacteria, wherein the gene list/profile is generated from elastic net. In one embodiment the profile according to the disclosure employed 75 or less such as 50 or less genes. In one embodiment the gene list is relevant to a virus, such as Influenza virus.

The present disclosure also extends to kits adapted to performing a method of the present disclosure, for example comprising probes for a minimal discriminatory gene list suitable for discriminating infection by a pathogen, for example a specific pathogen and optionally one or more house-keeping genes.

In another embodiment the method is used to distinguish specific inflammatory or other conditions such as Kawasaki disease, Stills disease, or SLE from other inflammatory or infectious conditions.

In one embodiment the kit comprises reagents and/or instructions for performing the method according to the present disclosure, for example reagents for fluorescence analysis or colorimetric analysis.

In one embodiment the present disclosure provides a method of providing a minimal discriminatory gene list, for example for infection by a pathogen, such as a specific pathogen comprising the steps of analysing data from gene expression analysis of cohorts of patients employing elastic net to generate a gene a list of discriminating genes.

In one embodiment the list of discriminatory genes is shown in table 1 and/or 2 below.

In one embodiment the method is used to determine a minimal discriminatory gene list for Influenza H1N1.

TABLE 1
Up-regulated transcripts in H1N1 relative to the controls

| Probe Ids | Coefficients | Log fold change | Weights |
|---|---|---|---|
| 610451 | 0.000752 | 1.805871 | 1 |
| 290730 | 0.000765 | 2.20636 | 1 |
| 2570300 | 0.00118 | 2.927905 | 1 |
| 3170136 | 0.001946 | 2.073891 | 1 |
| 3360343 | 0.00284 | 3.528729 | 1 |
| 3990010 | 0.004617 | 1.874574 | 1 |
| 160731 | 0.00577 | 1.200175 | 1 |
| 7160129 | 0.005875 | 0.758313 | 1 |
| 7610440 | 0.007521 | 2.280976 | 1 |
| 1440615 | 0.007542 | 3.660582 | 1 |
| 5960747 | 0.007657 | 1.358896 | 1 |
| 3990170 | 0.008631 | 5.569215 | 1 |
| 2030209 | 0.008769 | 1.344619 | 1 |
| 6650242 | 0.008786 | 2.448184 | 1 |
| 2120079 | 0.009914 | 1.889369 | 1 |
| 5700735 | 0.010185 | 1.620488 | 1 |
| 7550066 | 0.010715 | 0.761084 | 1 |
| 7040707 | 0.010821 | 1.283736 | 1 |
| 840068 | 0.011405 | 2.037187 | 1 |
| 6100022 | 0.013383 | 1.985805 | 1 |
| 4060358 | 0.013808 | 1.858927 | 1 |
| 1820592 | 0.015227 | 1.914044 | 1 |
| 630278 | 0.016108 | 1.989441 | 1 |
| 3440348 | 0.018891 | 0.470806 | 1 |
| 3830762 | 0.018948 | 1.036655 | 1 |
| 6860164 | 0.022342 | 1.639984 | 1 |
| 6650348 | 0.023525 | 0.689152 | 1 |
| 6250168 | 0.023745 | 0.502976 | 1 |
| 5490546 | 0.027791 | 0.921163 | 1 |
| 110437 | 0.031861 | 0.78173 | 1 |
| 460220 | 0.044505 | 0.166349 | 1 |

In one embodiment the Influenza (H1N1) gene profile comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 up-regulated genes of Table 1.

TABLE 2
Down-regulated transcripts in H1N1 relative to the controls

| Probe Ids | Coefficients | Log fold change | Weights |
|---|---|---|---|
| 5690431 | −0.094726508 | −0.753031126 | −1 |
| 4150113 | −0.061144707 | −0.159999494 | −1 |
| 1690630 | −0.054162779 | −0.839486391 | −1 |
| 2680072 | −0.052067018 | −0.821493239 | −1 |
| 3940484 | −0.045045211 | −1.269576683 | −1 |
| 6860193 | −0.036248073 | −1.341402342 | −1 |
| 6770762 | −0.034806556 | −0.479136328 | −1 |
| 4760431 | −0.027576615 | −1.541706037 | −1 |
| 1400520 | −0.022885104 | −1.461132467 | −1 |
| 3710647 | −0.017738548 | −1.322730672 | −1 |
| 2490450 | −0.017023191 | −2.014506989 | −1 |
| 5700189 | −0.01574585 | −0.848395868 | −1 |
| 1190039 | −0.014448645 | −0.92742984 | −1 |
| 5670605 | −0.011893507 | −1.780690305 | −1 |
| 5570427 | −0.009051972 | −0.934088764 | −1 |
| 3850246 | −0.008852178 | −1.381246681 | −1 |
| 4220592 | −0.008167991 | −0.741474274 | −1 |
| 270168 | −0.008072421 | −0.965004156 | −1 |
| 4900731 | −0.007777538 | −1.196066773 | −1 |
| 7210082 | −0.007639308 | −1.160186905 | −1 |
| 3940458 | −0.005871574 | −0.489713401 | −1 |
| 3800735 | −0.005665939 | −1.169817853 | −1 |
| 5290482 | −0.001870834 | −0.954613281 | −1 |
| 4880360 | −0.001602126 | −1.380367932 | −1 |

In one embodiment the Influenza (H1N1) gene profile comprises 1, 2, 3, 4, 5, 6, 7, 8, 9, 10, 11, 12, 13, 14, 15, 16, 17, 18, 19, 20, 21, 22, 23, 24, down-regulated genes of Table 2.

In one embodiment the Influenza (H1N1) gene profile comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 up-regulated genes of Table 1 and 1, 2, 3, 4, 5, 6, 7, 8, 9, 10, 11, 12, 13, 14, 15, 16, 17, 18, 19, 20, 21, 22, 23, 24 down-regulated genes of Table 2.

In one embodiment the unweighted simple disease risk score for H1N1 vs control is 18. In one embodiment the weighted simple disease risk score for H1N1 vs control is 19. In one embodiment the weighted score has better discriminatory power for H1N1 versus controls.

In one embodiment there is provided an Influenza H1N1-specific gene expression profile comprising one or more genes with discriminatory power as defined herein, identified by the method of the present invention.

The present disclosure extends to each permutation and discloses the same directly and unambiguously, for 1 gene from table 1 and 1 gene from table 2, 1 gene from table 1 and 2 genes from table 2 and so on and so forth.

The probe ids in table 1 and table 2 correspond to specific genes. The reference to the probe may in the appropriate context be taken to be a reference to the corresponding gene.

TABLE 3
Probe IDs and their corresponding Illumina gene names.

| Illumina Probe ID | | Illumina Probe ID | |
|---|---|---|---|
| (up-regulated) | Illumina Gene | (down-regulated) | Illumina Gene |
| 610451 | HIST2H2AA3 | 5690431 | SNTA1 |
| 290730 | HIST1H2BD | 4150113 | MDM2 |
| 2570300 | IFI44 | 1690630 | FAM43A |
| 3170136 | SAMD9L | 2680072 | OLFM1 |
| 3360343 | RSAD2 | 3940484 | MEF2D |
| 3990010 | HS.125087 | 6860193 | RTN1 |
| 160731 | SHISA5 | 6770762 | TPPP3 |
| 7160129 | SBF2 | 4760431 | LOC136143 |
| 7610440 | XAF1 | 1400520 | CNTNAP2 |
| 1440615 | OTOF | 3710647 | MXD4 |
| 5960747 | TRIM22 | 2490450 | LOC91561 |
| 3990170 | IFI27 | 5700189 | TCTN1 |
| 2030209 | MTF1 | 1190039 | HLA-DPA1 |
| 6650242 | IFITM3 | 5670605 | MATK |
| 2120079 | EIF2AK2 | 5570427 | GLS |
| 5700735 | PARP9 | 3850246 | HOPX |
| 7550066 | MERTK | 4220592 | CACNA2D3 |
| 7040707 | KIF1B | 270168 | HLA-DRA |
| 840068 | C3AR1 | 4900731 | HLA-DMB |
| 6100022 | HIST2H2AC | 7210082 | EIF3F |
| 4060358 | ABCA1 | 3940458 | CRYL1 |
| 1820592 | HIST2H2AA3 | 3800735 | HVCN1 |
| 630278 | H1F0 | 5290482 | IFP38 |
| 3440348 | ASH2L | 4880360 | FBL |
| 3830762 | TMEM119 | | |
| 6860164 | CLEC1B | | |
| 6650348 | LAPTM4B | | |
| 6250168 | HS.549784 | | |
| 5490546 | SLC30A1 | | |
| 110437 | TERF1 | | |
| 460220 | ITGA1 | | |

In one embodiment the profile comprises all the genes from table 1 and all the genes from table 2.

Results of table 1 and 2 are from the elastic net variable selection on H1N1 vs control expression data.

Step 3: Conversion of Multi-Gene Transcript Disease Signatures into a Single Number Disease Score

Once the RNA expression signature of the disease has been identified by variable selection, the transcripts are separated based on their up- or down-regulation relative to the comparator group. The two groups of transcripts are selected and collated separately.

Step 4: Summation of Up-Regulated and Down-Regulated RNA Transcripts

To identify the single disease risk score for any individual patient, the raw intensities, for example fluorescent intensities (either absolute or relative to housekeeping standards) of all the up-regulated RNA transcripts associated with the disease are summated. Similarly summation of all down-regulated transcripts for each individual is achieved by combining the raw values (for example fluorescence) for each transcript relative to the unchanged housekeeping gene standards. Since the transcripts have various levels of expression and respectively their fold changes differ as well, instead of summing the raw expression values, they can be scaled and normalised between [0,1]. Alternatively they can be weighted to allow important genes to carry greater effect. Then, for every sample the expression values of the signature's transcripts are summated, separately for the up- and down-regulated transcripts.

The total disease score incorporating the summated fluorescence of up- and down-regulated genes is calculated by adding the summated score of the down-regulated transcripts (after conversion to a positive number) to the summated score of the up-regulated transcripts, to give a single number composite expression score. This score maximally distinguishes the cases and controls and reflects the contribution of the up- and down-regulated transcripts to this distinction.

Comparison of the Disease Risk Score in Cases and Controls

The composite expression scores for patients and the comparator group may be compared, in order to derive the means and variance of the groups, from which statistical cut-offs are defined for accurate distinction of cases from controls. Using the disease subjects and comparator populations, sensitivities and specificities for the disease risk score may be calculated using, for example a Support Vector Machine and internal elastic net classification.

Development of the Disease Risk Score into a Simple Clinical Test for Disease Severity or Disease Risk Prediction

The approach outlined above in which complex RNA expression signatures of disease or disease processes are converted into a single score which predicts disease risk can be used to develop simple, cheap and clinically applicable tests for disease diagnosis or risk prediction.

The procedure is as follows: For tests based on differential gene expression between cases and controls (or between different categories of cases such as severity), the up- and down-regulated transcripts identified employing step 2 above may be printed onto a suitable solid surface such as microarray slide, bead, tube or well.

Up-regulated transcripts may be co-located separately from down-regulated transcripts either in separate wells or separate tubes. A panel of unchanged housekeeping genes may also be printed separately for normalisation of the results.

RNA recovered from individual patients using standard recovery and quantification methods (with or without amplification) is hybridised to the pools of up- and down-regulated transcripts and the unchanged housekeeping transcripts.

Control RNA is hybridised in parallel to the same pools of up- or down-regulated transcripts.

Total value, for example fluorescence for the patient-sample and optionally the control sample is then read for up- and down-regulated transcripts and the results combined to give a composite expression score for patients and controls, which is/are then compared with a reference range of a suitable number of healthy controls or comparator patients.

Correcting the Detected Signal for the Relative Abundance of RNA Species in the Patient Sample

Step 2 above explains how a complex signature of many transcripts can be reduced to the minimum set that is maximally able to distinguish between patients and other phenotypes. For example, within the up-regulated transcript set, there will be some transcripts that have a total level of expression many fold lower than that of others. However, these transcripts may be highly discriminatory despite their overall low level of expression. The weighting derived from the elastic net coefficient can be included in the test, in a number of different ways. Firstly, the number of copies of individual transcripts included in the assay can be varied. Secondly, in order to ensure that the signal from rare, important transcripts are not swamped by that from transcripts expressed at a higher level, one option would be to select probes for a test that are neither overly strongly nor too weakly expressed, so that the contribution of multiple probes is maximised. Alternatively, it may be possible to adjust the signal from low-abundance transcripts by a scaling factor.

Whilst this can be done at the analysis stage using current transcriptomic technology as each signal is measured separately, in a simple colorimetric test only the total colour change will be measured, and it would not therefore be possible to scale the signal from selected transcripts. This problem can be circumnavigated by reversing the chemistry usually associated with arrays. In conventional array chemistry, the probes are coupled to a solid surface, and the amount of biotin-labelled, patient-derived target that binds is measured. Instead, we propose coupling the biotin-labelled cRNA derived from the patient to an avidin-coated surface, and then adding DNA probes coupled to a chromogenic enzyme via an adaptor system. At the design and manufacturing stage, probes for low-abundance but important transcripts are coupled to greater numbers, or more potent forms of the chromogenic enzyme, allowing the signal for these transcripts to be ‘scaled-up’ within the final single-channel colorimetric readout. This approach would be used to normalise the relative input from each probe in the up-regulated, down-regulated and housekeeping channels of the kit, so that each probe makes an appropriately weighted contribution to the final reading, which may take account of its discriminatory power, suggested by the weights of variable selection methods.

The detection system for measuring multiple up or down regulated gens may also be adapted to use rTPCR to detect the transcripts comprising the diagnostic signature, with summation of the separate pooled values for up and down regulated transcripts, or physical detection methods such as changes in electrical impedance. In this approach, the transcripts in question are printed on nanowire surfaces or within microfluidic cartridges, and binding of the corresponding ligand for each transcript is detected by changes in impedance or other physical detection system

### EXAMPLE

Experimental Validation of this Approach

In order to validate the approach for converting complex RNA expression signatures into a single individual patient risk score, we utilised a microarray study comparing the RNA expression profiles of patients with H1N1 influenza infection with that of healthy controls and a range of other bacterial and viral infections. Expression analysis was undertaken on Illumina HT12-v3 microarrays according to standard protocols.

Patient Groups

Over the winter of 2009-10, 165 acutely ill febrile children (below 17 years) presenting to St Mary's Hospital, London UK were recruited to the study. As the clinical spectrum of H1N1/09 was unknown at the time of study commencement, a broad case definition was adopted for recruitment in order to capture the full spectrum of H1N1/09 manifestations. This approach ensured that we were able to recruit patients with H1N1/09 or with other febrile illnesses, both bacterial and viral. Patients were recruited as early as possible in their hospital assessment, before any diagnostic studies were available, encompassing a wide spectrum of clinical presentations consistent with influenza infection.

Research samples for RNA expression were collected concurrently with clinical diagnostic samples, and patients were later assigned to diagnostic categories once the microbiological and virological studies became available. Children with co-morbidities likely to have strong effects on gene expression were excluded from the study (bone marrow transplant recipients and children on chemotherapy).

Based on diagnostic bacterial and viral test results, patients were assigned to pathogen specific groups: 29 patients had H1N1/09 infection (including 6 with multiple pathogen infection) and 39 children had Respiratory Syncytial Virus (RSV) infection (including 16 with multiple pathogens). The RSV cohort represented the largest single virus-infected comparator group. A further 103 children had a spectrum of other acute respiratory infections, including 32 children with confirmed bacterial infection. Of these, 21 patients had a gram-positive organism (S.pneumoniae in 15, S.pyogenes in 4, S.aureus in 2). Forty-two children without RSV or H1N1/09 infection had one or more of the following detected: rhinovirus or enterovirus (n=29), bocavirus (8), parainfluenza (5), adenovirus (5), influenza A H3N2 (2), metapneumovirus (1), gram-negative bacterial infection (7). 11 children with on-going chemotherapy or previous bone marrow transplant were excluded from further analysis, as was 1 child with H1N1/09 and RSV co-infection. 39 control children were recruited at the time of having blood tests; 3 of these had recent infections or vaccinations (within 3 weeks) and were excluded. Twenty-five H1N1/09 patients without RSV or bacterial co-infection had samples for RNA analysis, six of whom had co-infection with one or more non-RSV viruses. Twelve patients were classified as ‘severe’, 5 of whom died.

Pathogen Diagnosis.

Viral diagnostic testing was undertaken on nasopharyngeal aspirates using immunofluorescence (RSV, adenovirus, parainfluenza virus, influenza A+B) and nested PCR (RSV, coronavirus, adenovirus, parainfluenza 1-4, influenza A+B, bocavirus, metapneumovirus, rhinovirus). Bacterial diagnostics included culture of blood and pleural fluid, and pneumococcal antigen detection in blood or urine where available.

RNA Expression Profiling:

Whole blood was collected in PAXgene® tubes and RNA extracted using PAXgene blood RNA extraction kits (Qiagen) according to the manufacturer's instructions. After quantification and quality control, biotin-labelled cRNA was prepared from 330 ng mRNA using Ilium ina Total Prep RNA Amplification kits (Applied Biosystems). 750 ng labelled cRNA was hybridised to Illumina HumanHT-12 v3 Expression BeadChips, and the microarrays scanned. Quality control parameters were assessed using Genome Studio software and visual inspection of the microarray images. The effects of age, gender and technical batch were removed using linear regression.

Microarray Analysis

Expression data were analysed using ‘R’ Language and Environment for Statistical Computing 2.12.1 and GeneSpringGX 11.5 software (Agilent). Mean raw intensity values for each probe were corrected for local background intensities, and quantile normalised. The dataset was filtered to exclude probes that were flagged as ‘present’ on less than 90% of the arrays in at least one group of interest. Expression values were transformed to a log₂ scale.

The hypothesis that the expression level for each probe differed between comparator patient groups was assessed using Welch's moderated t-test (20). P values were adjusted using Benjamini and Hochberg's method to control for the false discovery rate (17). For each comparison of interest the most significant probes were selected, based on P value and fold-change >2.

We compared each infection cohort to controls to derive a list of significantly DE transcripts for each comparison with P<0.05 and log₂ FC>1 (Table 4). When comparing the transcriptional response of two infection cohorts, we included the union of DE transcripts between healthy controls and either pathogen.

The Support Vector Machine (SVM) method for supervised learning was used to classify patients into groups, based on our pre-defined signatures. We applied a linear SVM to define a hyperplane in a high-dimensional transformed feature space that maximally discriminated two patient groups. We used leave-one-out cross-validation to calculate the classification accuracy.

The data have been deposited in NCBI's Gene Expression Omnibus (Edgar et al., 2002) and are accessible through Series accession number GSE42026 (http://www.ncbi.nlm.nih.gov/geo/query/acc.cgi?acc=GSE42026).

TABLE 4
Demographic and clinical data of recruited subjects

| | H1N1/09 | RSV | Bacterial | Controls | P value |
|---|---|---|---|---|---|
| Sex M:F (% male) | 12:13 | 21:13 | 8:13 | 18:15 | NS |
| | (48) | (62) | (38) | (55) | |
| Age (years): median (IQR) | 4.0 | 0.4 | 1.9 | 3.4 | P < 0.0001 |
| | (1.6-7.5) | (0.1-1.4) | (1.0-4.4) | (1.5-6.9) | |
| Days from symptoms to | 5 | 4 | 3.5 | N/A | NS |
| recruitment median (IQR) | (3.0-7.0) | (2.0-6.3) | (2.0-10.5) | | |
| Number of patients | 25^{a} | 34^{a} | 18 | 33 | N/A |
| No co-infection | 19 | 23 | 13^{b} | N/A | |
| Co-infection | 6 | 11 | 5 | | |
| Bocavirus | 5 | 5 | 0 | | |
| Rhinovirus | 2 | 4 | 0 | | |
| Adenovirus | 0 | 2 | 1 | | |
| Seasonal flu parainfluenza | 0 | 1 | 1 | | |
| Metapneumo | 0 | 0 | 1 | | |
| RSV | 0 | 0 | 2 | | |
| H1N1/09 | (1)^{a} | N/A | N/A | | |
| S. pneumoniae | N/A | 0 | N/A | | |
| S. pyogenes | (1)^{a} | (2)^{a} | 12 | | |
| S. aureus | 0 | 0 | 4 | | |
| | 0 | 0 | 2 | | |
| Deaths | 5 | 0 | 1 | N/A | NS |
| Pathogen cohort for arrays | 19 (without | 22 (without | 18 (excludes | 33 | N/A |
| | co-infection) | co-infection) | H1N1, RSV) | | |
| Lymphocyte proportion (array | 0.21 | 0.39 | 0.17 | 0.45 | P < 0.001 |
| patients): median (IQR) | (0.10-0.32) | (0.28-0.49) | (0.08-0.25) | (0.38-0.56) | for HvsC |
| Neutrophil proportion (array | 0.69 | 0.47 | 0.74 | 0.45 | P < 0.001 |
| patients): median (IQR) | (0.52-0.84) | (0.40-0.64) | (0.64-0.87) | (0.35-0.51) | for HvsC |
| Monocyte proportion (array | 0.04 | 0.09 | 0.03 | 0.07 | NS |
| patients): median (IQR) | (0.01-0.08) | (0.03-0.15) | (0.0-0.08) | (0.06-0.09) | |

NS—not significant (corrected P < 0.05); IQR—interquartile range; N/A—not applicable.
^{a}Two patients each in the H1N1/09 and RSV cohorts with confounding co-infections (RSV or bacterial) were excluded from array analysis and from demographic calculations.
^{b}After excluding patients with H1N1/09 or RSV, patients with confirmed gram-positive bacterial infection were analysed irrespective of other viral co-infection - no virological investigations were available for 9 bacterial infection patients recruited outside the pandemic period.

The gender distribution between cohorts was not different. The ages of the H1N1/09, bacterial and control cohorts were not significantly different. The RSV cohort was younger, as expected for RSV bronchiolitis admissions. Days from symptom onset to recruitment, and deaths in each cohort were not significantly different. Lymphocyte proportion was lower, and neutrophil proportion higher (denominator total leucocytes) in H1N1/09 patients than controls, but was not significantly different when compared to the RSV or bacterial groups.

Pathogen-Specific Signatures Versus Controls

Comparison of 19 patients with H1N1/09 mono-infection versus controls using modified T-tests derived 1,267 transcripts matching a significance threshold of p<0.001 after multiple testing correction. Unsupervised clustering using this set separated cases and controls into distinct highly concordant groups. The validity of the 1,267 transcript set was assessed using the Support Vector Machine approach, which returned a very strong classification accuracy of 96% on both mono-infected H1N1/09 patients and patients with non-RSV viral co-infections, indicative of the dominance of the influenza signature over other viruses.

We also found highly concordant clustering of cases and controls for the RSV (mono-infection) and gram-positive bacterial patients (with or without coincident non-H1N1 non-RSV viral infection), with respectively 1,172 and 1,869 differentially expressed probes identified for p<0.001. The validity of these probe sets was supported by SVM leave-one-out validation with an accuracy of 95% and 98% for RSV and bacterial patients respectively.

An independent statistical validation of the pathogen-control signatures was undertaken using the elastic net variable selection method on all valid transcripts to derive a minimal probe set best able to distinguish the pathogen and control cohorts, irrespective of degree of fold change. This method identified 40 transcripts distinguishing H1N1/09 and controls (8).

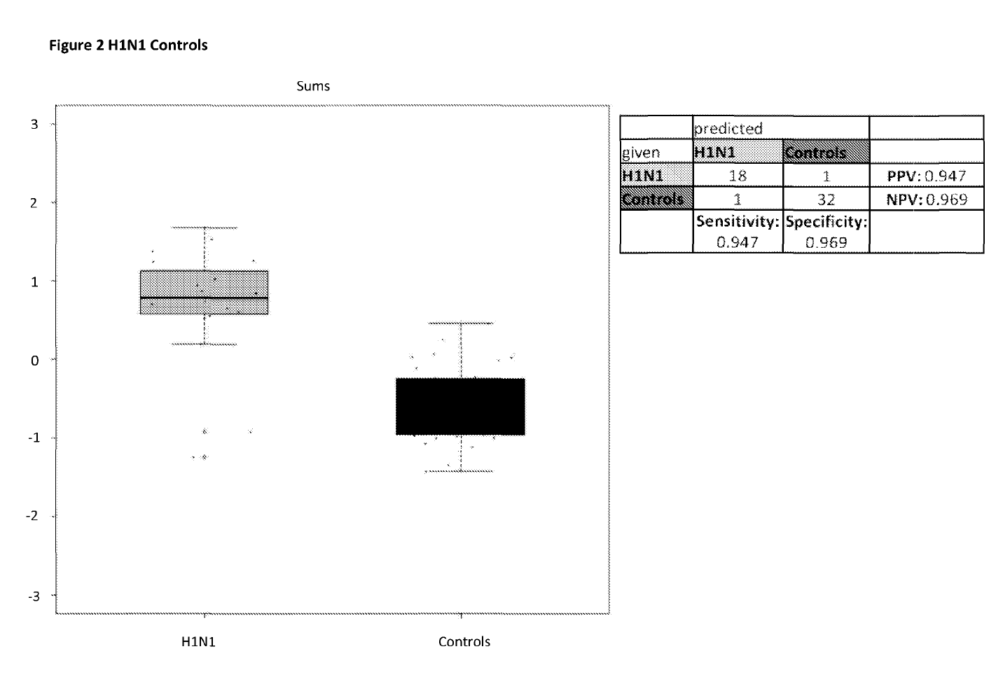

In order to convert the complex multi gene signature into a single disease risk score for individual patients we followed the procedure described in methods above in which up-regulated gene transcripts were identified (see Table 1) and the individual fluorescence of all up-regulated probes summated; then the down-regulated transcripts (Table 2) and their transcripts were summated to give a total fluorescence score for up- and down-regulated genes. These were combined to give a single score for each individual patient and each individual control population. FIG. 2 displays the disease risk score for patients and controls with box and whiskers indicating 25^{th} and 75^{th} percentile distribution of the data. We calculated sensitivity and specificity for distinction of cases from controls using the single value disease risk score and a Support Vector Machine with 10 fold cross validation and found a sensitivity of 94% and specificity of 96%.

Weighting of the Transcripts to Improve Discrimination.

In order to improve the discrimination we used the coefficient from the elastic net analysis to weight each up- and down-regulated gene (Table 1 and Table 2). We then repeated the summation of up- and down-regulated genes, and found improved discrimination and sensitivity and specificity.

Application of the Method to Distinguish RSV Infection from H1N1

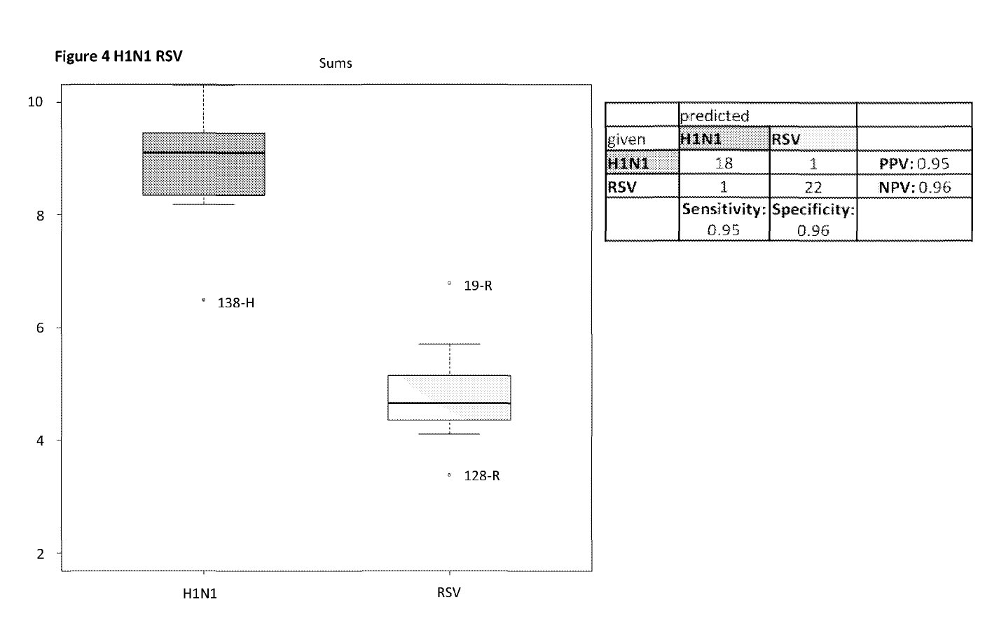

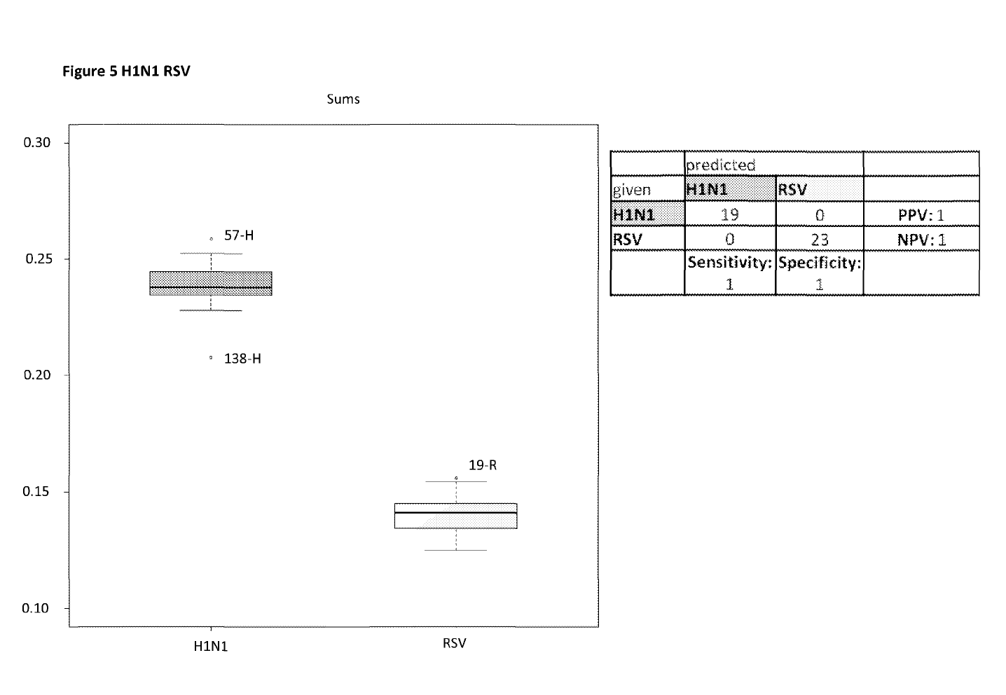

In order to explore the wider applicability of the method we used elastic net variable selection to identify a 100 gene signature which distinguished H1N1 patients from those with RSV infection. As shown in FIG. 4, the summation of total fluorescence provided good discrimination of the two patient cohorts. Furthermore, weighting of the transcripts using the elastic net coefficient improved the discrimination further (FIG. 5).

Application to Other Bacterial and Viral Infections

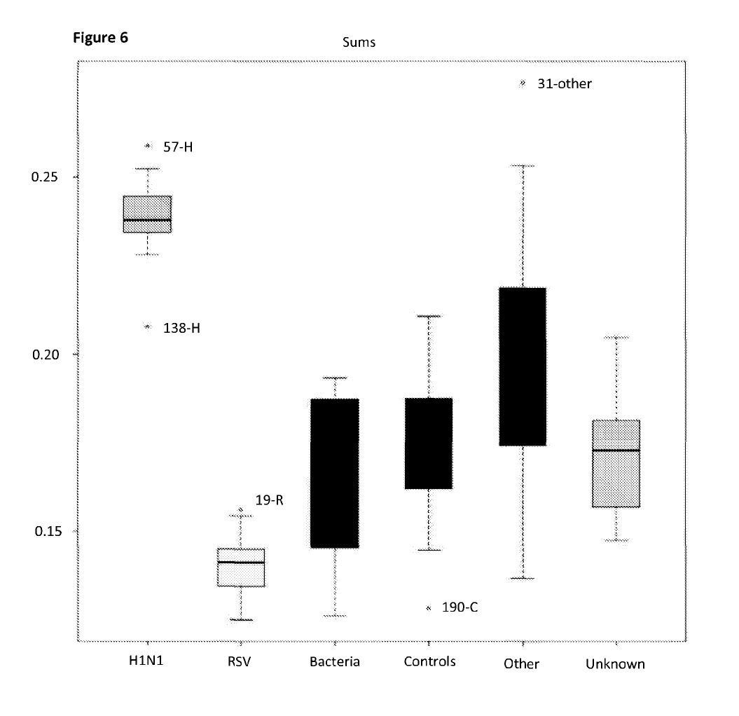

In order to provide further evidence that our approach can be generalised to other infections we repeated the analysis described to compare H1N1 with RSV infection. We used the same set of probes to calculate total fluorescence for H1N1 vs patients with bacterial infection, patients with a range of other viral infections, and patients with severe illness without identified bacterial or viral infections (FIG. 6). For each comparison H1N1 could be distinguished from the other bacterial and viral infections

Conclusions

These data provide proof of concept that complex signatures of RNA expression can be converted into a simple diagnostic score for each patient, by combining the expression values for a small number of carefully selected up- and down-regulated transcripts. The result can be derived without the need for complex bioinformatic analysis. Application of weighting using the coefficients identified by elastic net improves the discriminatory power, and we propose a methodology to translate this weighting into a simple diagnostic platform using the adaptation of readily available colorimetric techniques. Our methodology has potential for use in simple diagnostic tests requiring minimal bioinformatic analysis and suitable for development as clinical tools for diagnosis of a wide range of infectious, inflammatory, malignant or genetic conditions.

### REFERENCES

1. Griffiths, M. J., Shafi, M. J., Popper, S. J., Hemingway, C. A., Kortok, M. M., Wathen, A., Rockett, K. A., Mott, R., Levin, M., Newton, C. R., et al. 2005. Genomewide analysis of the host response to malaria in Kenyan children. The Journal of Infectious Diseases 191:1599-1611.

2. Pathan, N., Hemingway, C. A., Alizadeh, A. A., Stephens, A. C., Boldrick, J. C., Oragui, E. E., McCabe, C., Welch, S. B., Whitney, A., O'Gara, P., et al. 2004. Role of interleukin 6 in myocardial dysfunction of meningococcal septic shock. Lancet, The 363:203-209.

3. Kampmann, B., Hemingway, C., Stephens, A., Davidson, R., Goodsall, A., Anderson, S., Nicol, M., Schölvinck, E., Relman, D., Waddell, S., et al. 2005. Acquired predisposition to mycobacterial disease due to autoantibodies to IFN-gamma. The journal of clinical investigation 115:2480-2488.

4. Ramilo, O., Allman, W., Chung, W., Mejias, A., Ardura, M., Glaser, C., Wittkowski, K. M., Piqueras, B., Banchereau, J., Palucka, A. K., et al. 2007. Gene expression patterns in blood leukocytes discriminate patients with acute infections. Blood 109:2066-2077.

5. Berry, M. P., Graham, C. M., McNab, F. W., Xu, Z., Bloch, S. A., Oni, T., Wilkinson, K. A., Banchereau, R., Skinner, J., Wilkinson, R. J., et al. 2010. An interferon-inducible neutrophil-driven blood transcriptional signature in human tuberculosis. Nature 466:973-977.

6. Baehner, F. L., Lee, M., Demeure, M. J., Bussey, K. J., Kiefer, J. A., and Barrett, M. T. 2011. Genomic signatures of cancer: basis for individualized risk assessment, selective staging and therapy. J Surg Oncol 103:563-573.

7. Allantaz, F., Chaussabel, D., Stichweh, D., Bennett, L., Allman, W., Mejias, A., Ardura, M., Chung, W., Wise, C., Palucka, K., et al. 2007. Blood leukocyte microarrays to diagnose systemic onset juvenile idiopathic arthritis and follow the response to IL-1 blockade. J Exp Med 204:2131-2144.

8. Zou, H., and Hastie, T. 2005. Regularization and variable selection via the elastic net. J Roy Stat Soc Ser B 67:301-320.

9. R Development Core Team (2006). R: A language and environment for statistical computing. R Foundation for Statistical Computing, Vienna, Austria. ISBN 3-900051-07-0, URL http://www.R-project.org)

10 Jean (ZHIJIN) Wu and Rafael Irizarry with contributions from James MacDonald Jeff Gentry (2005). gcrma: Background Adjustment Using Sequence Information. R package version 2.4.1.

11. Wu Z, Irizarry R A, Gentleman R, Martinez-Murillo F, Spencer F: A model-based background adjustment for oligonucleotide expression arrays. Journal of the American Statistical Association 2004, 99:909-917.

12. Peter Warren (2005). panp: Presence-Absence Calls from Negative Strand Matching Probesets. R package version 1.2.0. 5. R. Gentleman, V. Carey and W. Huber (2006). genefilter: genefilter: filter genes. R package version 1.10.1.

13. Jain N, Thatte J, Braciale T, Ley K, O'Connell M, Lee J K. Local-pooled-error test for identifying differentially expressed genes with a small number of replicated microarrays. Bioinformatics. 2003 Oct. 12; 19(15): 1945-51.

14. Nitin Jain, Michael O'Connell and Jae K. Lee. Includes R source code contributed by HyungJun Cho <hcho@virginia.edu> (2006). LPE: Methods for analyzing microarray data using Local Pooled Error (LPE) method. R package version 1.6.0. http://www.r-proiect.org.

15. Smyth, G. K. Linear models and empirical Bayes methods for assessing differential expression in microarray experiments. Statistical Applications in Genetics and Molecular Biology (2004) 3, No. 1, Article 3.

16. Smyth, G. K. (2005). Limma: linear models for microarray data. In: ‘Bioinformatics and Computational Biology Solutions using R and Bioconductor’. R. Gentleman, V. Carey, S. Dudoit, R. Irizarry, W. Huber (eds), Springer, New York, pages 397-420 10. Baldi P, Long A D. A Bayesian framework for the analysis of microarray expression data: regularized t-test and statistical inferences of gene changes. Bioinformatics. 2001 June; 17(6):509-19.

17. Benjamini, Y. and Hochberg, Y. (1995) Controlling the false discovery rate: a practical and powerful approach to multiple testing. J. Roy. Stat. Soc. B., 57, 289-300.

18. Katherine S. Pollard, Yongchao Ge and Sandrine Dudoit. multtest: Resampling-based multiple hypothesis testing. R package version 1.10.2.

19. Jess Mar, Robert Gentleman and Vince Carey. MLInterfaces: Uniform interfaces to R machine learning procedures for data in Bioconductor containers. R package version 1.4.0. 15. Soukup M, Cho H, and Lee J K (2005). Robust classification modeling on microarray data using misclassification penalized posterior, Bioinformatics, 21 (Suppl): i423-i430. 16. Soukup M and Lee J K (2004). Developing optimal prediction models for cancer classification using gene expression data, Journal of Bioinformatics and Computational Biology, 1(4) 681-694.

20. Welch B L. The generalization of ‘Students’ problem when several different population variances are involved. Biometrika 1947; 34:28-35.

21. Irizarry R A, Hobbs B, Collin F, Beazer-Barclay Y D, Antonellis K J, Scherf U, Speed T P. Exploration, normalization, and summaries of high density oligonucleotide array probe level data. Biostatistics. 2003 April; 4(2):249-64.

22. Tusher, Virginia Goss; Tibshirani, Robert; Chu, Gilbert (2001). “Significance analysis of microarrays applied to the ionizing radiation response”. Proceedings of the National Academy of Sciences of the United States of America 98 (18): 5116-5121.

1. A method of processing gene expression data generated from analysis of a patient-sample, for establishing the presence of a signature indicative of infection by a pathogen or other specific disease state, such as an inflammatory disease, a chronic disease or malignant condition which is defined by specific clinical diagnostic criteria, comprising the steps:
a) optionally normalising and/or scaling numeric values of the gene expression data
b) taking the normalised and/or scaled numeric values or the raw numeric values, each of which comprise both positive and/or negative numeric values and designating all said numeric values to be negative or alternatively all positive,
c) optionally refining the discriminatory power of one or more up-regulated genes and down-regulated genes by statistically weighting some of the numeric values associated therewith, and
d) summating the positive or negative numeric values obtained from step b) or step c) to provide a composite expression score,
wherein the composite expression score obtained from step d) is compared to a control and the comparison allows the sample to be designated as positive or negative for the relevant infection.

2. A method according to claim 1, wherein the gene expression data is generated from analysis of a microarray.

3. A method according to claim 1, wherein the gene expression data is in the form of a fluorescence reading.

4. A method according to claim 1, wherein the gene expression data is in the form of a colorimetric reading.

5. A method according to claim 1, wherein the pathogen is viral, bacterial, parasitic or fungal.

6. A method according to claim 1, wherein the patient sample is from a febrile patient.

7. A method of claim 6, wherein the method is performed to establish if the fever is associated with a bacterial or viral infection.

8. A method of diagnosing an inflammatory, a malignant or a chronic condition with defined clinical diagnostic criteria, comprising a method of processing gene expression data generated from analysis of a patient-sample comprising the steps:
a) optionally normalising and/or scaling numeric values of the gene expression data
b) taking the normalised and/or dcaled numeric values or the raw numeric values, each of which comprise both positive and/or negative numeric values and designating all said numeric values to be negative or alternatively all positive,
c) optionally refining the discriminatory power of one or more up-regulated genes and down-regulated genes by statistically weighting some of the numeric values associated therewith, and
d) summating the positive or negative numeric values obtained from step b) or step c) to provide a composite expression score,
wherein the composite expression score obtained from step d) is compared to a control and the comparison allows the sample to be designated as positive or negative for the relevant infection.

9. A method according to claim 1, which further comprises the step of amplifying RNA from the patient-sample

10. A method according to claim 1, which further comprises the step of quantifying RNA from the patient-sample.

11. A kit of parts for performing the method of claim 1, comprising a reagent, control and/or device for identifying a predetermined profile indicative of a pathogenic infection or other specific disease such as an inflammatory, a malignant or chronic disease.

12. A kit according to claim 12, wherein the device is an array device consisting of genes of the profile and optionally house-keeping genes.

13. A method according to claim 1, wherein the composite expression score is a composite expression score for Influenza H1N1.

14. An Influenza H1N1 specific gene expression profile comprising modulation of one or more genes with discriminatory power from Table 1 and/or Table 2.

15. An Influenza H1N1 specific gene expression profile according to claim 14, comprising all the genes of Table 1 and Table 2.
